(19) United States
(12) Patent Application Publication
(10) Pub. No.: US 2011/0314977 A1
(43) Pub. Date: Dec. 29, 2011

(54) TUBE PURGING INSTRUMENT

(76) Inventor: Howell G. Lewis, Tallahassee, FL (US)

(21) Appl. No.: 12/962,192

(22) Filed: Dec. 7, 2010

Related U.S. Application Data
(63) Continuation-in-part of application No. 11/970,968, filed on Jan. 8, 2008, now abandoned.

(51) Int. Cl. A61M 39/28

(52) U.S. Cl. 81/486

(57) ABSTRACT

A tube purging instrument, device has cooperating rollers which can be compressed and/or clamped together upon a flexible tube to compress the tube partially or completely between the rollers and which can then be rolled together along the length of the tube in order to advance the contents of the tube in the direction of movement of the rollers. The rollers are preferably equipped with flanges or rims which fit over an outer diameter of a tube to maintain registration of the rollers upon tube as the rollers travel in Counter-rotation along a length of the tube.

### RELATED APPLICATIONS

This application is a continuation-in-part from U.S. patent application Ser. No. 11/970,908 filed on Jan. 8, 2008, which is incorporated herein by reference in its entirety.

### FIELD OF THE INVENTION

The present invention is in the general field of tools, instruments and devices, including hand tools and instruments and more particularly hand tools and instruments and devices which can be used in connection with fluid and material handling equipment, and with medical equipment and procedures and devices.

### BACKGROUND OF THE INVENTION

In many different types of medical procedures drainage tubing is used to convey body fluids away from the body and to a receptacle. Common examples are open and invasive surgical procedures within major body cavities, such as for example the peritoneum, including all types of thoracic and vascular surgical procedures, following which continuous drainage of fluids, such as saline and lymphatic fluids, is required for normal healing processes. In some procedures such as cardiac bypass there may be multiple drainage sites at which receiving ends of drainage tubes are positioned. Each drainage tube must be kept sterile, and establish a positive flow direction away from the body, by gravity or siphon, and be continuously monitored for efficacy throughout the healing process. One common product or device which is used for surgical drainage is the Jackson-Pratt or IP drain, which is a suction and drainage device used to pull excess fluid from the body. The JP drain consists of a flexible plastic bulb which is connected to a flexible drainage tube which extends from the body. Patients or caretakers can milk or strip the drains by compressing the tube near the point of exit from the body and sliding the compression point down the length of the tube to the drainage receptacle or bulb. This task cannot be accomplished by patients who are unconscious or disabled, and must therefore be performed by a nurse or other caregiver. Purging, stripping or “milking” drainage tube contents is commonly done manually, by holding one end of the tube with one hand distant from the receptacle, and sliding compressed fingers of the other hand down the length of the tube toward the receptacle. This two-handed act is awkward, unsanitary and not practical or easy to be performed by the patient. There is also risk of dislodging the drainage tube from the exit at the body and disrupting surrounding sutures. Although principally described in the context of medical application and use, the device and instrument of the disclosure and the invention is equally applicable and effective in any environment wherein the purging or movement of tube contents is required, including without limitation veterinary medicine, laboratory experiments and operations, commercial and research chemical or physical operations, and any tube transfer or conveyance of liquid, flowable solids or gases.

### SUMMARY OF THE INVENTION

The disclosure and invention provides a new and improved device and instrument and method for purging or stripping or voiding the contents of a flexible tube. In a representative embodiment of the principles and concepts of the disclosure, a tube purging instrument has first and second rollers, each roller mounted for rotation upon a spindle, each spindle extending from an arm, each arm being movable with respect to the other arm in order to change relative positions of the rollers, the rollers being positionable by movement of the arms so that the spindles are generally parallel and rolling surfaces of the rollers are closely adjacent whereby a segment of a tube positioned between the rolling surfaces of the rollers is compressed and counter-rotation of the rollers against the tube is caused by movement of the arms in a path generally parallel to a length of the tube.

These and other embodiments and aspects of the disclosure and exemplary embodiments are further described herein with reference to the accompanying drawing Figures.

### DESCRIPTION OF THE DRAWINGS

FIG. 1 is a perspective view of a tube purging instrument of the disclosure in a representative mode of use.

FIG. 2 is a perspective view of a tube purging instrument of the disclosure in a representative mode of use.

FIG. 3 is a profile view of a tube purging instrument of the disclosure.

FIG. 4 is a perspective view of a tube purging instrument of the disclosure.

FIG. 5 is a profile view of a tube purging instrument of the disclosure.

FIG. 6 is a profile view of a tube purging instrument of the disclosure.

FIG. 7 is a profile view of a tube purging instrument of the disclosure.

FIG. 8 is a perspective view of a tube purging instrument having a pivot casing.

FIG. 9 is an exploded view of the tube purging instrument of FIG. 8.

FIG. 10 is a cross-sectional view of the tube purging instrument of FIG. 8 in a first position in the direction of arrows 10-10.

FIG. 11 is a cross sectional view of the tube purging instrument of FIG. 8 in a second position.

### DETAILED DESCRIPTION OF PREFERRED AND ALTERNATE EMBODIMENTS

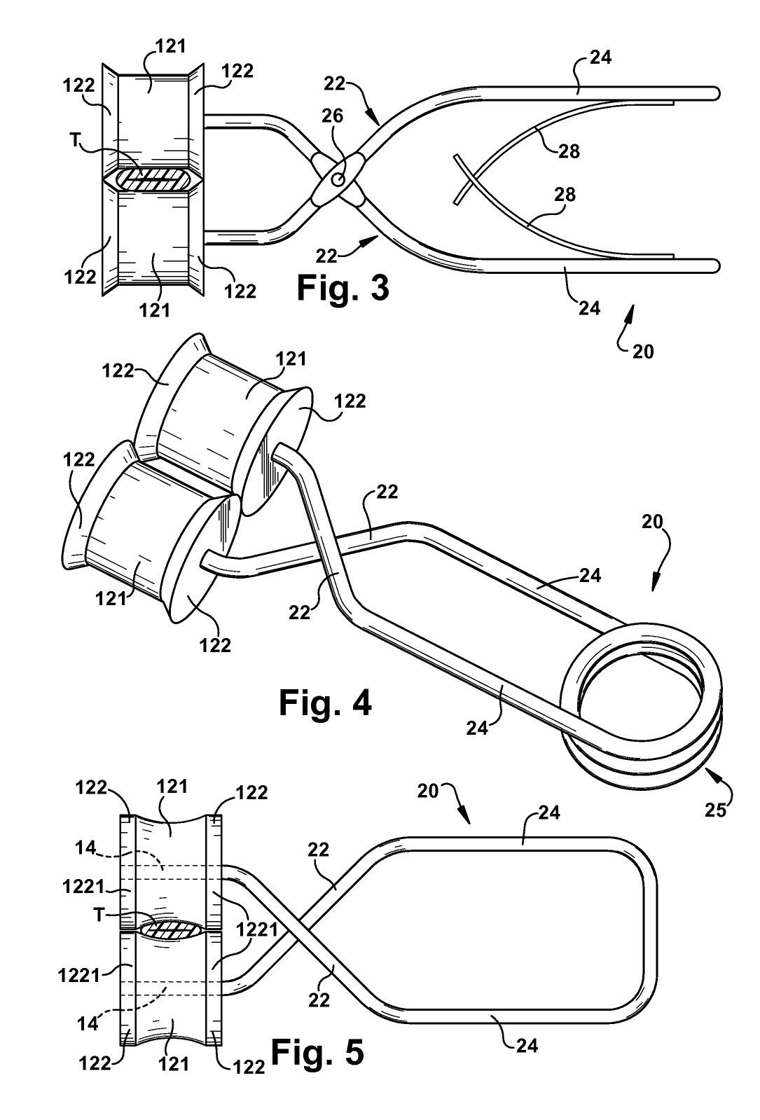

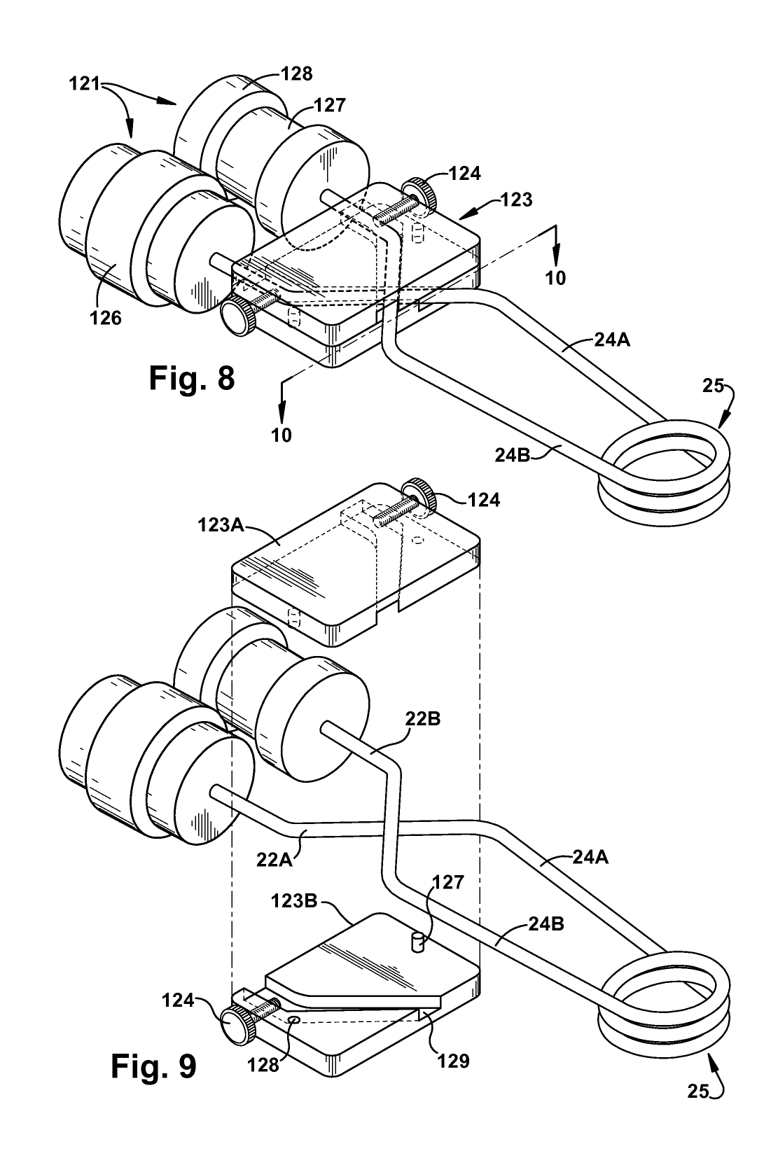

A tube purging instrument or device, shown in the Figures and indicated in its entirety at 10, is in the general form of a single hand-held and operated instrument, by, for example, a right or left hand, for use in connection with one or more tubes which are flexible to a degree which allows for transverse compression across a width of the tube(s) for partial or total occlusion of the tubal passage. An exemplary application and use of the tube purging instrument 10 is in connection with tubing used in medical application and procedures and drainage, such as the tubes of Jackson-Pratt drains, for transport of fluid and viscous material to or from the body. A common example is surgical drainage from the situs or cavity of surgical activity, where there has been invasion and insult to tissue or removal of tissue or organs. In order to quickly and efficiently purge a tube of contents in a sterile and safe manner, the device 10 is equipped with a pair of cooperating rollers 12 which are mounted for free rotation upon axles or spindles 14 which extend from a hand grip structure 20 as further described. The rollers 12 are configured to fit or extend over a tube of conventional size, such as according to the exterior outer diameter of the tube T. Each roller 12 has a rolling surface 121 which has a width which is preferably approximately equal to an outer diameter or width of tube T. Each roller 12, or optionally only one of the rollers 12, is preferably equipped with flanges or rims 122 at opposite axial ends of the roller 12 which straddle the tube T when the rolling surfaces 121 are aligned with the tube T, as shown in FIGS. 3, 5 and 6. With the rollers 12 in the fully clamped or compressed position, the opposing rims 122 are preferably in rolling contact or near contact bilaterally with respect to the tube T. This maintains the rollers 12, and particularly the rolling surfaces 121 of the rollers 12 in alignment with the tube T, so that the rollers 12 can be quickly advanced by rolling along the length of the tube T without concern for rolling off of the tube T. The rims 122 maintain the rolling surfaces 121 in linear alignment and registration with the tube T. As shown in the Figures, the rolling surfaces 121 of the rollers 12 may be cylindrical with a straight edge profile, or tapered as shown in FIG. 5 such as convex to conform to the exterior of a compressed cylindrical tube. The rims 122 may be tapered from the rolling surface 121 (FIGS. 2-4), offset radial for a tongue and groove formation (FIG. 8) or truncated (FIGS. 5-7) to have their own bearing surfaces 1221 which create contact between the two rollers.

Each roller 12 is mounted upon an axle or spindle 14 which extends from a hand grip structure 20, which has cooperating and opposing arms 22, each with an associated handle or gripping bar 24. The arms 22 cross at some point along the length of the structure 20 so that the action of squeezing the handles or gripping bars 24 together causes the rollers 12 to diverge to allow for positioning of the tube T between the rollers 12 and to be placed into contact with the rolling surfaces 121 of the rollers 12 to fully or substantially compress tube T, as shown for example in FIGS. 3, 5 and 6.

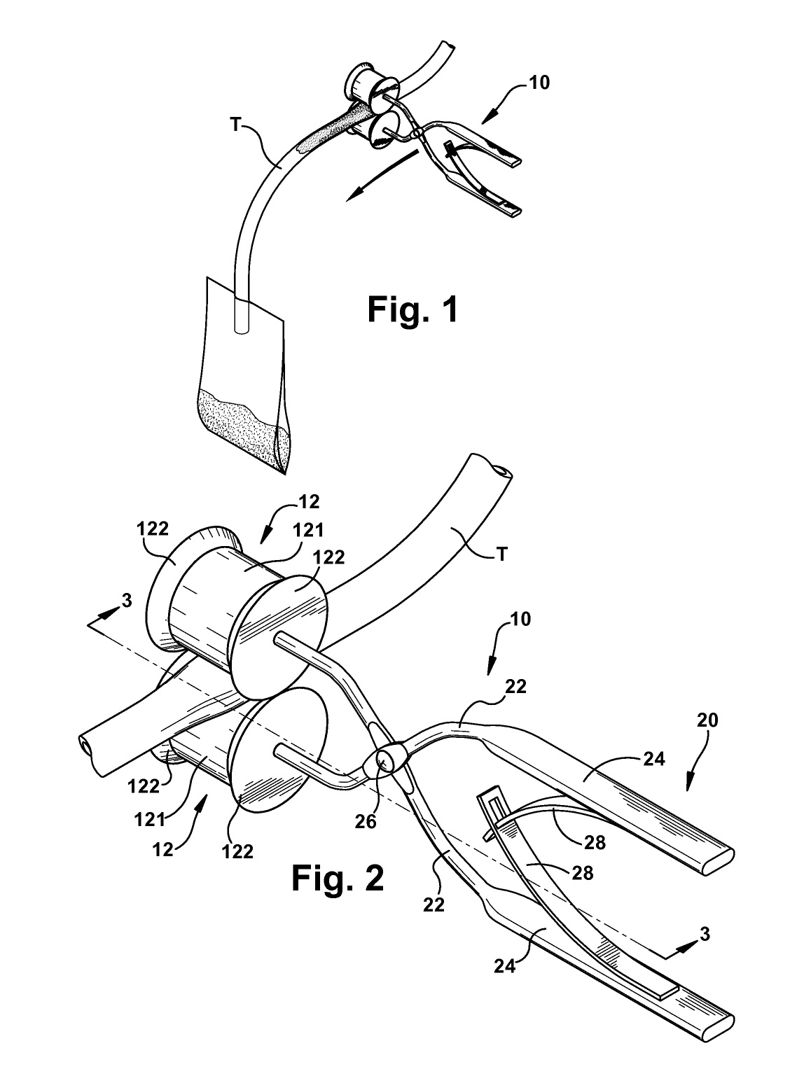

FIGS. 1-3 and 6 illustrate a scissors type embodiment of the hand grip structure 20 wherein the arms 22 are joined together by a pivot 26 about which and by which the described divergence and convergence of the rollers 12 occurs. The arms 22 can be biased toward the operative or compressed position with the rollers 12 (and axles 14) generally parallel, by the provision of a spring or spring system, such as the cooperating leaf springs 28 shown between the handles 24 in FIGS. 1-3, or a coil spring 25 as shown in FIG. 4, or by the inherent spring properties of the material such as spring steel as maybe used in the construction of a wire-form hand grip structure 20 as shown in FIG. 5.

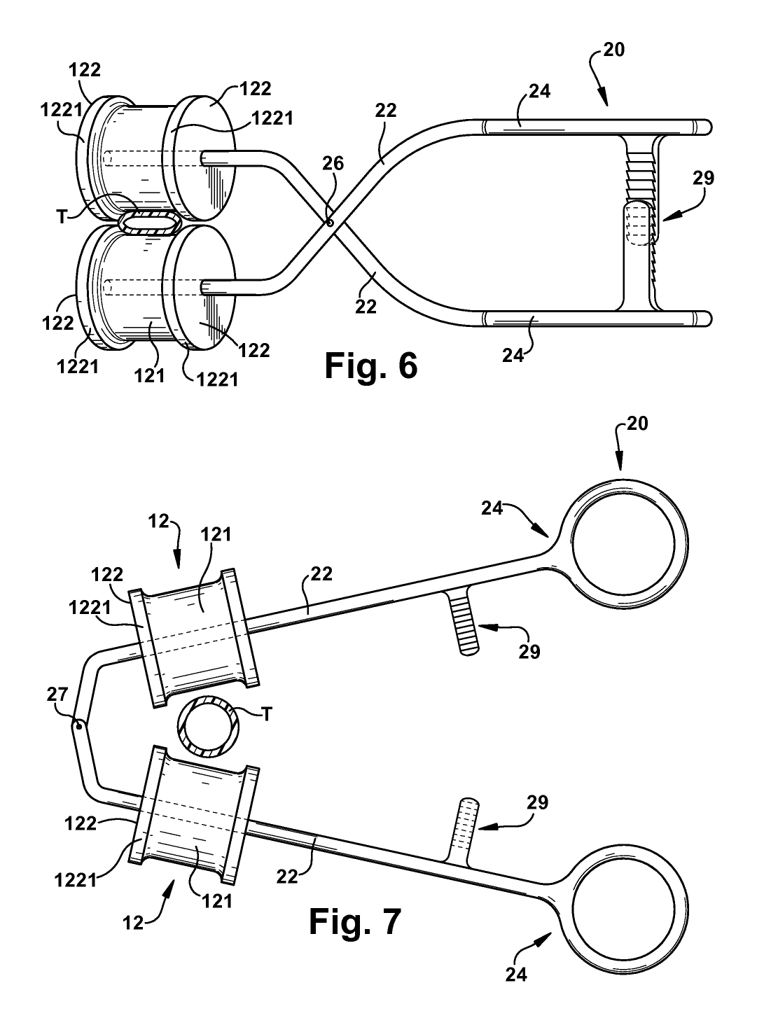

FIG. 6 illustrates an alternate embodiment of a hand grip structure 20 which also incorporated a pivot 26 connection between cooperating arms 22, and a clamp 29 located between the handles 24, which when engaged holds the rollers 12 in the tube-compressing position. The clamp 29 can be configured with multiple position settings, by the numerous cooperating and engaging teeth 291, to adjust the degree of compression placed upon the tube T by the rollers 12.

FIG. 7 illustrates an alternate embodiment of a hand grip structure 20 wherein the cooperating arms 22 Are hinged or pivoted at a pivot 27 which is located at a distal end of the arms 22 and distant from handles 24 with the rollers 12 located intermediate the pivot 27 and handles 24. The clamping and compressive action of the rollers 12 upon tube T is applied by manipulation of the handles 24, and held in such position by a similar clamp 29 as described in connection with the embodiment of FIG. 6.

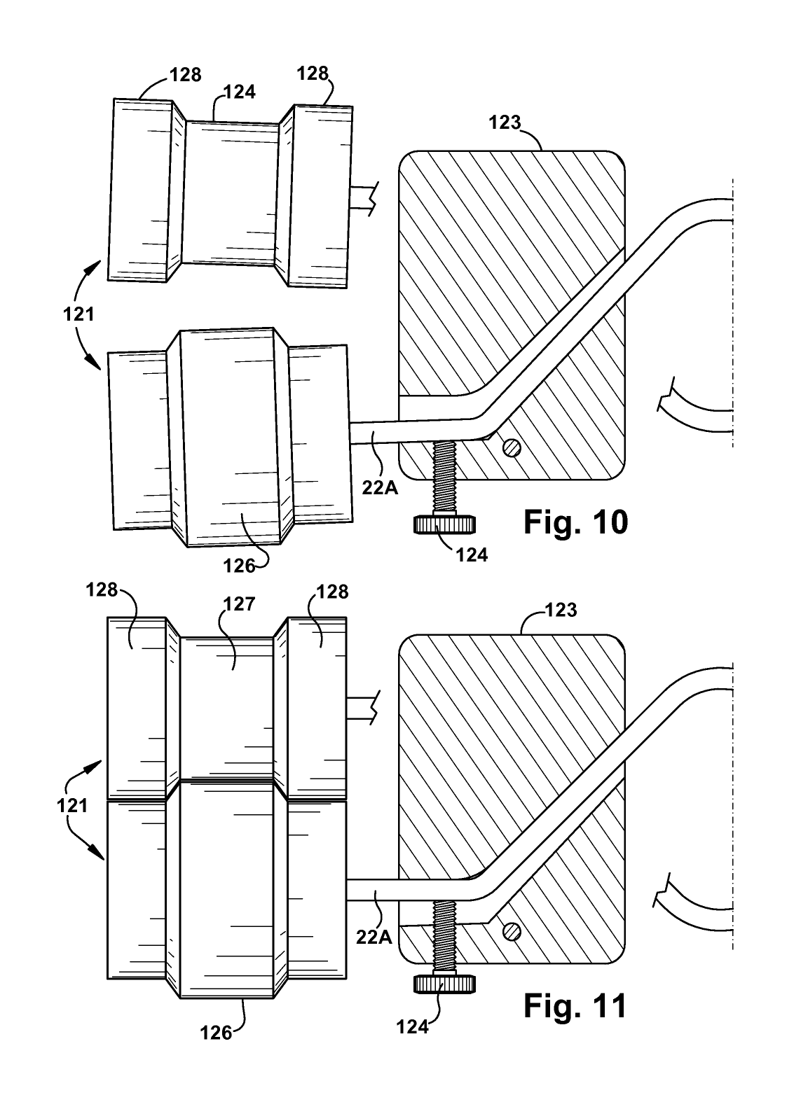

A preferred embodiment of the hand grip structure disclosed herein is shown in FIGS. 8-11. This embodiment includes a pair of cooperating arms 22A, 22B which are connected to a pair of handles 24A, 24B which in turn are connected to a coil spring 25, similar to the embodiment shown in FIG. 4. The preferred embodiment additionally contains a pivot casing 123 which surrounds the arms at the point of intersection. The arms 22A, 22B can be biased toward the operative or compressed position with the rollers 121 generally parallel by provision of the coil spring 25. The rollers 121 are offset radially, creating a tongue and groove configuration wherein one roller contains a projecting rib 126 and the other contains a channel 127 within which to accommodate the projecting rib 126 from the opposing roller. The outer walls 128 of the channel 127 prevent a tube from becoming dislodged from the device while in use. The two-piece pivot casing 123 surrounds the point of intersection between the opposing arms and prevents the arms 22A, 22B from opening due to radial torque. The pivot casing 123 works in combination with one or more tension screws 124, which when tightened, create additional force between the rolling surfaces 126, 127 by holding an arm 22A, 22B in position within the pivot casing. The additional force may be applied using the tensioning screw, thus eliminating the need to apply additional physical force. An exploded view of the two-piece pivot casing 123 is shown in FIG. 9. A first piece or component 123A of the pivot casing 123 contains a channel 129 for accommodating a first aura 22A of the device 10 at the point of intersection between the two arms 22. The second piece or component 123B of the pivot casing 123 is a mirror image of the first component 123A, having a channel 129 for accommodating the second arm 22B. Each of the two components of the pivot casing 123 contain an aperture or an opening accessible through the side of the component proximate to each channel 129. The opening leads to the channel 129 such that a tension screw 124 may be inserted therein to apply various amounts of pressure directly to the cooperating arms 22A, 22B located within each channel 129. The width of each channel 129 is greater than the width of each of the arms 22A, 22B, allowing for various positions of the rollers in relation to one another. For example, by securing the pivot casing 123 about the arms 22A, 22B without applying tension to the arms 22A, 22B via the screws 124, the arms will rest against The outer wall of the channel 129, as shown in FIG. 10. Applying pressure to the arms 22A, 22B by advancing the tension screws 124 toward the arms forces each arm away from the outside wall of the channel 129 and closer to the opposing arm, shown in FIG. 11, thereby decreasing the amount of space between the rollers 121. The arms, and therefore the rollers, can be positioned at various degrees of proximity with respect to each other by adjusting the position of the tension screws 124. The two components 22A, 22B of the pivot casing 123 are removably attached to one another via male and female attachment mechanisms. The first component 123A contains one male attachment mechanism 127 located proximate to one edge of the component and one female attachment mechanism 128 located proximate to the opposite edge of the component. The second component 123B contains exact male and female attachment mechanisms oppositely arranged with relation to attachment mechanisms of the first component 123A so that the male attachment mechanism of the first component 123A is inserted into the female attachment mechanisms of the second component 123B and vice versa. The components may be detached by applying opposing forces to each component. Any other suitable fastening means may be used to hold the first and second components 123A, 123B together.

The disclosure and invention in one or more of the various representative forms thus provides a useful instrument and device for rapid, sterile and efficient purging, stripping or milking of contents of flexible tubing, by the described application and use to any length of tubing in any application. The disclosure and invention is not limited to any particular dimensions or configurations, but for use with common tube sizes the instrument or device may be ergonomically configured and dimensioned for hand-held operation and use and convenient portage and storage.

1. A tube purging instrument comprising:
first and second rollers, each roller mounted for rotation upon a spindle, each spindle extending from an arm, each arm being connected to a coil spring and movable about a pivot with respect to the other arm in order to change relative positions of the rollers from a generally open position wherein the rollers are spread apart and a generally closed position where the rollers are relatively close together, the arms being spring-biased to the generally open position,
the rollers being positionable between the generally open position and the generally closed position by movement of the arms by compression of the arms together to place the rollers in the generally closed position and release of the arms to place the rollers in the generally open position wherein the spindles are generally parallel and roller surfaces of the rollers are closely adjacent and biased to the open position whereby a segment of a tube positioned between the rolling surfaces of the rollers is compressed by compressing the arms together and counter-rotation of the rollers against the tube is caused by movement of the rollers in a path generally parallel to a length of the tube;
the first roller having a protruding rib and the second roller having a channel therein which accommodates the protruding rib of the first roller;
a pivot casing which extends about and removably encases a portion of the arms, the pivot casing having at least one adjustment screw used to vary the distance between the rollers.

2. The tube purging instrument of claim 1 further comprising a handle connected to each of the arms.

3. The tube purging instrument of claim 2, wherein the arms and handles are spring-biased toward a position where the rollers are proximate and the spindles on which the rollers are mounted are generally parallel by a coil spring located between the arms.

4. The tube purging instrument of claim 3, wherein the coil spring is integral with the handles.

5. The tube purging instrument of claim 1, wherein the rolling surfaces. of the rollers have a generally flat profile.

6. The tube purging instrument of claim 1, wherein the rolling surfaces of the rollers have a generally curved profile.

7. The tube purging instrument of claim 1, wherein the rollers are configured to substantially compress a tube positioned between the rollers when the rollers are moved together by the arms or the pivot casing.

8. The tube purging instrument of claim 1, wherein the rolling surfaces contain radial grooves.

9. The tube purging instrument of claim 1, wherein a width of the rolling surface of each roller is equal to or greater than a width of a tube positioned between the rolling surfaces.

10. A tube purging instrument comprising:
a pair of generally opposed rollers, each roller mounted to a hand grip structure, the hand grip structure comprising a pair of arms, each arm having a spindle upon which one of the rollers is mounted for rotation, the arms being connected together by a coil spring for relative pivotal movement of the rollers between a spaced-apart generally open position and a proximate generally closed position wherein the rollers are generally axially aligned and the spindles are generally parallel, each of the rollers having a rolling surface configured for contact with a tube and at least one hearing surface configured for contact with the bearing surface of the opposed roller, and wherein the rolling surfaces of the rollers are opposed and closely spaced apart when the arms are in the generally closed position so as to be able to compress a segment of a tube positioned between surfaces of the rollers, and wherein the bearing surfaces of the rollers are in rolling contact, and wherein the arms of the hand grip structure are spring-biased by the coil spring connected to and integral with the hand grip structure to the generally open position wherein the rollers are spaced apart and the spindles are generally parallel;
wherein when the arms are in the generally closed position the bearing surface of one roller fits within a channel on the opposing roller so that the tube is secured between the two bearing surfaces within the channel.

11. The tube purging instrument of claim 10 further comprising a pivot casing which encloses the pivot and prevents the arms from opening due to radial torque.

12. The tube purging instrument of claim 11, wherein the pivot casing contains at least one tension screw at one end of the casing to provide additional force between the rolling surfaces of the rollers.

13. The tube purging instrument of claim 10, wherein the hand grip structure further comprises a handle associated with each of the arms, the handle extending from the pivot point and away from the rollers.

14. The tube purging instrument of claim 10, wherein the surface area of the bearing surfaces of each roller is substantially equal to the surface area of the tube compressed therebetween.

15. The tube purging instrument of claim 10, wherein the arms are configured to cross at the pivot.

16. The tube purging instrument of claim 10, wherein the rolling surfaces of the rollers are generally flat.

17. The tube purging instrument of claim 10, wherein the bearing surfaces contain radial grooves.

18. The tube purging instrument of claim 10, wherein one of the rollers contains a protruding rib.

19. The tube purging instrument of claim 10, wherein the pair of rollers are radially offset.
